(19) United States
(12) Patent Application Publication
(10) Pub. No.: US 2004/0181111 A1
(43) Pub. Date: Sep. 16, 2004

(54) Production of 1-butene

(75) Inventors: Marcus Sigl, Ellerstadt (DE); Christoph Ubler, Quirnheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Akiengesellschaft, Ludwigshafen (DE)

(21) Appl. No.: 10/773,169

(22) Filed: Feb. 9, 2004

(30) Foreign Application Priority Data
Mar. 14, 2003 (DE) 103 11 139.5

(51) Int. Cl. C07C 5/13

(52) U.S. Cl. 585/750

(57) ABSTRACT

Process for producing a 1-butene-containing C4-hydrocarbon stream (1-C₄⁼ stream) from a 1-butene- and 2-butene-containing C4-hydrocarbon stream (1- and 2-C₄⁼ feed stream) whose 1-butene content is lower than that of the 1-C₄⁼ stream, by

a) feeding the 1- and 2-C₄⁼ feed stream and a 1-butene- and 2-butene-containing C4-hydrocarbon stream (1- and 2-C₄⁼ recycle stream) whose 1-butene content is lower than that of the 1-C₄⁼ stream and which has been produced by means of step (b) below into a distillation column and taking off the 1-C₄⁼ stream and a 2-butene-containing C4-hydrocarbon stream (2-C₄⁼ stream) whose 1-butene content is lower than that of the 1- and 2-C₄⁼ feed stream and of the 1- and 2-C₄⁼ recycle stream from the distillation column (step a) and

b) producing the 1 - and 2-C₄⁼ recycle stream from the 2-C₄⁼ stream by bringing the 2-C₄⁼ stream into contact with an isomerization catalyst which catalyzes the conversion of 2-butenes into 1-butene in a reaction zone (step b).

The present invention relates to a process for producing a 1 -butene-containing C4-hydrocarbon stream (1-C₄⁼ stream) from a 1-butene- and 2-butene-containing C4-hydrocarbon stream (1- and 2-C₄⁼ feed stream) whose 1-butene content is lower than that of the 1-C₄⁼ stream, by

a) feeding the 1- and 2-C₄⁼ feed stream and a 1-butene- and 2-butene-containing C4-hydrocarbon stream (1- and 2-C₄⁼ recycle stream) whose 1-butene content is lower than that of the 1-C₄⁼ stream and which has been produced by means of step (b) below into a distillation column and taking off the 1-C₄⁼ stream and a 2-butene-containing C4-hydrocarbon stream (2-C₄⁼ stream) whose 1-butene content is lower than that of the 1- and 2-C₄⁼ feed stream and of the 1- and 2-C₄⁼ recycle stream from the distillation column (step a) and

b) producing the 1- and 2-C₄⁼ recycle stream from the 2-C₄⁼ stream by bringing the 2-C₄⁼ stream into contact with an isomerization catalyst which catalyzes the conversion of 2-butenes into 1-butene in a reaction zone (step b).

It is generally known that the isomerization of 2-butenes to 1-butene is an equilibrium reaction. cis-2-Butene, trans-2-butene and 1-butene are present in equilibrium with one another. The thermodynamic data are reported in D. Stull, “The Chemical Thermodynamics of Organic Compounds”, J. Wiley, New York 1969.

It is known from EP-A-751106 that 1-butene can be obtained from a C4-hydrocarbon stream by

a) subjecting the butene-containing hydrocarbon stream to a selective hydrogenation to eliminate multiply unsaturated hydrocarbons,

b) fractionally distilling the hydrocarbon stream obtained in step (a) to give a pure 1-butene fraction and a fraction comprising paraffins and 2-butenes,

c) removing the paraffins from the fraction comprising paraffins, 1-butene and 2-butenes by treatment with molecular sieves,

d) subjecting the fraction obtained in step c) to a double bond isomerization,

e) recirculating the fraction which has been subjected to the isomerization in step d) to step a) after it has been admixed with a fresh C₄-hydrocarbon.

The important difference between this and the process of the present invention is that the fraction subjected to the isomerization in step d) is recirculated to the hydrogenation step a) and not directly to the distillation step. A disadvantage of this is that the volume of the recycle stream is increased and the reactor in which the hydrogenation is carried out is burdened with compounds which are inert in respect of the hydrogenation and are removed only in the subsequent distillation.

WO 02/096843 describes a process for obtaining 1-butene from 2-butenes. Here, a hydrocarbon stream comprising mainly 2-butenes is firstly subjected to an isomerization and the reaction mixture formed is subjected to a distillation. In the distillation, a 1-butene-rich stream is separated from a 2-butene-rich stream and the latter is recirculated to the isomerization unit. However, this process is uneconomical for a hydrocarbon stream which contains significant amounts of 1-butene. In addition, a relatively large number of undesirable by-products are formed because of the presence of multiply unsaturated compounds in the isomerization step. As a result of the distillation being carried out subsequent to the isomerization stage, troublesome low-boiling constituents of the feed (e.g. butyne, butadienes, propadiene, propyne) get into the isomerization reactor and can there damage the catalyst or lead to the formation of undesirable by-products. In the process of the present invention, these low-boiling components are largely removed in the upstream distillation and do not get into the 2-C₄⁼ stream fed into the isomerization reactor.

It was an object of the present invention to provide a process by means of which the 1-butene content of C4-hydrocarbon streams can be increased at the expense of the proportion of 2-butenes in a particularly economical manner.

The 1- and 2-C₄⁼ feed stream is a C₄ fraction which generally has a butene content of from 30 to 100% by weight, preferably from 40 to 98% by weight, particularly preferably from 50 to 95% by weight. Apart from the butenes, it is possible for up to 10% by weight, preferably up to 5% by weight, of multiply unsaturated compounds or alkynes, especially those having three or four carbon atoms, e.g. butadienes, butynes, vinylacetylene, propyne and propadiene, to be additionally present in the 1- and 2-C₄⁼ feed stream. Furthermore, from 0.5 to 60% by weight, preferably from 1 to 50% by weight, of C4-alkanes and isobutene may also be present. Further hydrocarbons having more than 5 carbon atoms, in particular pentanes and pentenes, may be present in amounts up to 10% by weight.

In the present text, the generic term “butenes” is used only for linear butenes and does not encompass isobutene.

Particularly useful feed streams are raffinates (raffinate I or raffinate II).

Such raffinates I can be produced by

subjecting naphtha or other hydrocarbon compounds to a steam cracking or FCC process and taking off a C₄-hydrocarbon fraction from the stream formed and

producing a C₄-hydrocarbon stream (raffinate 1) consisting essentially of isobutene, 1-butene, 2-butenes and butanes from the C₄-hydrocarbon fraction by selectively hydrogenating the butadienes and butynes to butenes or butanes or removing the butadienes and butynes by extractive distillation.

Furthermore, the raffinates I are obtainable by

producing a C₄-olefin mixture from a butane-containing hydrocarbon stream by dehydrogenation and subsequent isolation of the C₄-olefins and

producing a C₄-hydrocarbon stream (raffinate I) consisting essentially of isobutene, 1-butene, 2-butenes and butanes from the C₄-olefin mixture by selectively hydrogenating the butadienes and butynes to butenes or butanes or removing the butadienes and butynes by extractive distillation.

The raffinate II can be produced from the raffinate I by separating off most of the isobutene from the raffinate I by known chemical, physicochemical or physical methods.

In a third method, raffinate 11 can be obtained by preparing a C₄-olefin mixture from methanol by dehydrogenation (MTO process) and, if appropriate, freeing this of butadienes or alkynes by distillation, partial hydrogenation or extractive distillation.

For further purification, the raffinate 11 can be freed of catalyst poisons by treatment with adsorbent materials.

The isomerization of 2-butenes to 1-butene is limited by the thermodynamic equilibrium of the n-butene isomers. The proportion of 1-butene in the thermodynamic equilibrium increases at high temperatures. The maximum yields of 1-butene (2-butene conversion x selectivity) which can be achieved in a single pass through the reactor are limited by the thermodynamic equilibrium to about 14% at 200° C. and about 29% at 500° C. The yields quoted are based on the thermodynamic data published in D. Stull “The Chemical Thermodynamics of Organic Compounds”, J. Wiley, New York, 1969. The process of the present invention is therefore particularly economical in the case of a 1- and 2-C₄⁼ feed stream whose 1-butene content is higher than the equilibrium concentration of 1-butene when carried out at from 100 to 700° C., preferably from 200 to 500° C. For this reason, use is generally made of 1- and 2-C₄⁼ feed streams in which the ratio of 2-butenes to 1-butene is from 6:1 to 0.1:1, preferably from 3:1 to 0.2:1.

The distillation takes place in an apparatus suitable for this purpose, e.g. a bubble cap tray column, a column containing random packing, a column containing ordered packing or a dividing wall column. The distillation column preferably has from 30 to 80 theoretical plates, particularly preferably from 40 to 75 theoretical plates. The reflux ratio is generally from 10 to 50. The distillation is generally carried out at a pressure of from 5 to 20 bar.

Due to the low boiling point of 1-butene compared to the 2-butenes, the 1-C₄⁼ stream will be taken off in the upper part of the column, preferably at the top of the column. The 1-butene content, based on the sum of 1-butene and 2-butenes in the 1-C₄⁼ stream, is usually from 80 to 99.99%.

The 1- C₄⁼ stream particularly preferably comprises from 60 to 99.9% by weight of 1-butene and 2-butenes, from 0.01 to 10% by weight of multiply unsaturated compounds, e.g. butadienes, and from 0.01 to 40% by weight of compounds selected from the group consisting of isobutane, n-butane and isobutene.

The multiply unsaturated compounds can originate from the 1- and 2-C₄⁼ stream and they are also formed in step b) under certain conditions, in particular when particular catalysts are chosen.

The content of 2-butenes in the 1-C₄⁼ stream is reduced by from 20 to 99.99% compared to the 1- and 2-C₄⁼feed stream.

The 2-C₄⁼ stream is advantageously taken off in the lower part of the distillation column, preferably in the lower fifth of the distillation column, particularly preferably at the bottom of the column or at a point not more than five theoretical plates above this.

The content of 2-butenes, based on the sum of 2-butenes and 1-butene in the 2-C₄⁼ stream, is usually from 85 to 99.9%.

The content of 2-butenes in the 1- and 2-C₄⁼ recycle stream is usually reduced by from 5 to 30%, based on its content in the 2-C₄⁼ stream.

To avoid accumulation of high-boiling components, e.g. n-butane and hydrocarbon compounds having 5 and more carbon atoms, in the 1- and 2-C₄⁼ recycle stream, it will generally be necessary to take off a substream consisting essentially of 1-butene, 2-butenes, n-butanes and hydrocarbons having 5 and more carbon atoms at the bottom of the distillation column. However, it is likewise possible to discharge only part of the 2-C₄⁼ stream for this purpose. In this case, the 2-C₄⁼ stream is taken off at the bottom.

The content of 2-butenes in the C₄⁺ bottom stream, based on the sum of 2-butenes and 1-butene, is usually from 90 to 99.9%.

The content of 2-butenes in the C₄⁺ bottom stream is usually increased by up to 10%, based on its content in the 2-C₄⁼ stream. The size of the C₄⁺ bottom stream and its 2-butene content depends on the conversion of 2-butenes into 1-butene, which is generally from 70 to 99%, based on the content of 2-butenes in the 1- and 2-C₄⁼ feed stream.

In step b), the 2-C₄⁼ stream is passed over a customary isomerization catalyst. The choice of the isomerization catalyst is not restricted further; it only has to be able to effect the isomerization of 2-butenes to 1-butene. For example, it is possible to use basic catalysts or catalysts based on zeolites for this purpose; the isomerization can also be carried out under hydrogenating conditions over catalysts comprising noble metals.

Suitable catalysts include alkaline earth metal oxides on aluminum oxide, as described in EP-A 718036, mixed aluminum oxide/silicon oxide supports doped with oxides of the alkaline earth metals, boron group metals, lanthanides or elements of the iron group (U.S. Pat. No. 4,814,542) or γ-aluminum oxide containing alkali metals, as described in JP 51-108691. Furthermore, catalysts comprising manganese oxide on aluminum oxide, as described in U.S. Pat. No. 4,289,919, catalysts comprising magnesium oxide, alkali metal oxides and zirconium oxides dispersed on an aluminum oxide support, as described in EP-A 234498, and aluminum oxide catalysts which additionally contain sodium oxide and silicon oxide, as described in U.S. Pat. No. 4,229,610, are also useful.

Suitable zeolite-based catalysts are described in EP-A 129899 (zeolites of the pentasil type). Also suitable are molecular sieves exchanged with alkali metals or alkaline earth metals (as described in U.S. Pat. No. 3,475,511), aluminosilicates (as described in U.S. Pat. No. 4,749,819) and zeolites in alkali metal form or alkaline earth metal form (as described in U.S. Pat. No. 4,992,613) and those based on crystalline borosilicates (as described in U.S. Pat. No. 4,499,326).

The catalysts are usually used in a fixed bed, fluidized bed or moving bed. In practical operation, it has been found that the amount of 2-C₄⁼ stream passed over the catalyst per unit time should be from 0.1 to 40 g of 2-C₄⁼ stream/[g of catalyst×h].

The isomerization is preferably carried out in a fixed-bed reactor system through which the stream to be isomerized flows continuously. Suitable reactors are tube reactors, shell-and-tube reactors, tray reactors, coil reactors or helical reactors. The reaction is endothermic. Temperature control can be carried out in a customary fashion. In addition, the reaction can also be carried out in an adiabatic reaction system.

The 2-C₄⁼ stream can be taken off from the column in gaseous or liquid form. If the 2-C₄⁼ stream is liquid, it has to be vaporized prior to the reaction. The apparatus used for the vaporization is subject to no particular restriction. Customary vaporizer types such as natural convection vaporizers or forced circulation vaporizers are suitable.

Before the gaseous 2-C₄⁼ stream reaches the reaction zone of step b), it has to be heated to the reaction temperature. Heating can be carried out using the apparatuses customarily employed, e.g. plate heat exchangers or shell-and-tube heat exchangers.

The isomerization is carried out at a temperature at which a shift in the double bond is achieved while cracking processes, skeletal isomerizations, dehydrogenations and oligomerizations are largely avoided. The reaction temperature is therefore generally from 100 to 700° C., preferably from 200 to 600° C., particularly preferably from 200 to 500° C. The pressure is set so that the 2-C₄⁼ stream is present in gaseous form. It is generally from 0.1 to 40 bar, preferably from 1 to 30 bar, particularly preferably from 3 to 20 bar.

The 1- and 2-C₄⁼ recycle stream produced by means of the above-described isomerization is recirculated to the distillation column at a suitable point. It can be fed into the column in gaseous or liquid form. If the temperature difference between the reactor outlet and the column temperature at the height of the return point is large, it can be useful to cool the output from the reactor. Cooling or condensation is carried out by generally customary methods.

In a specific embodiment, the heat streams for vaporization and heating are combined with the heat streams for cooling and condensation. Such heat integration enables the energy consumption for the reaction unit to be minimized.

With time, carbon-containing compounds can deposit on the isomerization catalyst used for the reaction and these can lead to deactivation of the catalyst. Burning off these deposits makes it possible to increase the activity of the catalyst again. The burn-off process can be carried out in a separate apparatus or preferably in the apparatus used for the reaction. In a specific embodiment, the reactor is designed in duplicate so that one apparatus is alternately available for the reaction while the regeneration is carried out in the other apparatus. In the burn-off process, a nitrogen/oxygen mixture is passed over the catalyst. The volume ratio of nitrogen to oxygen is from 1 to 20% by volume of oxygen. The oxygen content of the mixture can alter during the regeneration process. A low oxygen content is typically employed at the beginning and this is then increased. This makes it possible for the quantity of heat generated by the exothermic burn-off process to be controlled. The regeneration is carried out at elevated temperature, typically at from 300 to 90° C., preferably from 350 to 800° C., particularly preferably from 400 to 700° C.

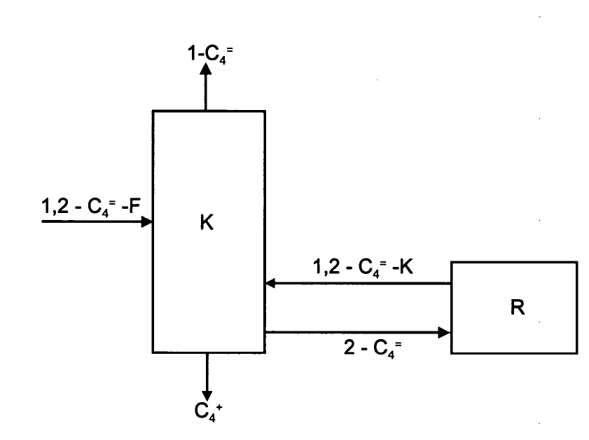

FIG. 1 schematically shows an apparatus for carrying out the process of the present invention using a column (K) and a reactor containing a fixed bed of an isomerization catalyst (R). The 1- and 2- C₄⁼ feed stream (1,2-C₄⁼—F) is fed into the column (K). The 1-C₄⁼ stream (1-C₄⁼) is taken off at the top and the stream C₄⁺ is taken off at the bottom. The 2-C₄⁼ stream (2-C₄⁼) is taken off from the column as a sidestream and is fed into the reactor (R). The reaction mixture formed there, viz. the 1,2-C₄ recycle stream (1,2-C₄⁼—K), is taken off from the reactor R and returned to the column (K).

The 1-C₄⁼ stream is particularly useful for the preparation of 3-hexene by metathesis. For this purpose, the 1-C₄⁼ stream is brought into contact with a customary metathesis catalyst at from 20 to 350° C. Such metathesis catalysts are generally known and are described, for example, in EP-A-1 134271. They are generally compounds of a metal of transition group VIb, VIIb or VIII of the Periodic Table of the Elements.

If the 1-C₄⁼ stream comprises alkynes or multiply unsaturated compounds, it is advisable to free the 1-C₄⁼ stream of these compounds by subjecting it to a selective hydrogenation in the presence of a palladium-containing catalyst in which virtually no conversion of 1-butene into 2-butenes occurs. Such a selective hydrogenation with avoidance of isomerization can be achieved by bringing the 1-C₄⁼ stream into contact with a catalyst bed comprising a supported palladium catalyst at from 40 to 60° C. and a hydrogen partial pressure of from 0.5 to 10⁶ pascal. This type of hydrogenation is generally known and is described, for example, in the monograph Petrochemical Processes, Volume 1, Synthesis-Gas Derivates and Major Hydrocarbons, A. Chauvel, G. Lefebvre, L. Castex, Institut Francais du Petrol Publications, 1989, Editions Technip, 27 Rue Ginoux, 75737 Paris, Cedex 15, on pages 208 and 209.

A 1-butene-rich C4 stream produced by the above-described process can also be used as starting material for many reactions. Examples which may be mentioned are: dimerization, oligomerization, epoxidation, carbonylation and copolymerization with ethylene.

Experimental Part

### EXAMPLE 1

An apparatus as shown in FIG. 1 is used for carrying out the experiment. A bubble cap tray column (K) having 85 trays and an internal diameter of 15 mm is employed. The pressure in the column is 8 bar, the temperature at the bottom is 71° C. and the temperature at the top is 63° C. The feed 1,2-C₄⁼—F is fed in at the level of tray 55, 1-C₄⁼ is taken off at the top of the column and the 2-butene-rich stream (2-C₄⁼) is 15 taken off at tray 5 and fed into the isomerization reactor (R). The feed leaving the isomerization reactor (R), viz. 1,2-C₄⁼—K, is fed back into the column at the level of tray 35. A high-boiling fraction (C₄⁺) is taken off at the bottom of the column. The amount and composition of the individual streams is shown in table 1. The isomerization is carried out at 250° C. and 6 bar over a zeolite catalyst (Na-ZBM-11). The catalyst volume is 3 dm³.

TABLE 1

| Stream | Unit | 1,2-C₄⁼-F | 1-C₄⁼ | C₄⁺ | 2-C₄⁼ | 1,2-C₄⁼-K |
|---|---|---|---|---|---|---|
| from | — | — | K | K | K | R |
| to | — | K | — | — | R | K |
| amount | kg/h | 0.650 | 0.607 | 0.043 | 3.259 | 3.259 |
| isobutane | g/g | 0.0250 | 0.0268 | 0.0000 | 0.0000 | 0.0000 |
| n-butane | g/g | 0.1250 | 0.1154 | 0.2984 | 0.3105 | 0.3110 |
| 1-butene | g/g | 0.4150 | 0.7922 | 0.0348 | 0.0427 | 0.1078 |
| c-2-butene | g/g | 0.2075 | 0.0027 | 0.2814 | 0.2631 | 0.2259 |
| t-2-butene | g/g | 0.2075 | 0.0390 | 0.3797 | 0.3830 | 0.3539 |
| isobutene | g/g | 0.0200 | 0.0214 | 0.0001 | 0.0001 | 0.0002 |
| density | kg/m³ | 594 | 535 | 535 | 8 | 8 |
| Temperature | ° C. | 25.0 | 62.7 | 71.4 | 250.0 | 245.2 |
| pressure | bar | 8.5 | 8.0 | 8.2 | 6.0 | 6.0 |

### EXAMPLE 2

The experiment is carried out in a manner analogous to example 1, except that the isomerization reactor is operated at 400° C. using a basic catalyst (SrO on γ-Al₂O₃, 4.2% by weight of Sr), the feed originates from a butane/butene separation and the product from the top is obtained in a purity of 99.9% by weight of 1-butene/Σ2-butenes. For this case, a bubble cap tray column (K) having 115 trays and an internal diameter of 55 mm is employed. The pressure in the column is 8 bar, the temperature at the bottom is then 71° C. and the temperature at the top is 63° C. The feed 1,2-C₄⁼—F is fed in at the level of tray 42, 1-C₄⁼ is taken off at the top of the column and the 2-butene-rich stream (2-C₄⁼) is taken off at tray 5 and fed into the isomerization reactor (R). The feed leaving the isomerization reactor (R), viz. 1,2-C₄⁼—K is fed back into the column at the level of tray 27. A high-boiling fraction (C₄⁺) is taken off at the bottom of the column. The mass flows and compositions of the individual streams are shown in table 2.

TABLE 2

| | | | | | | 1,2- |
|---|---|---|---|---|---|---|
| Stream | Unit | 1,2-C₄⁼-F | 1-C₄⁼ | C₄⁺ | 2-C₄⁼ | C₄⁼-K |
| from | — | — | K | K | K | R |
| to | — | K | — | — | R | K |
| amount | kg/h | 0.650 | 0.621 | 0.029 | 3.236 | 3.236 |
| isobutane | g/g | 0.000 | 0.0000 | 0.000 | 0.000 | 0.000 |
| n-butane | g/g | 0.0300 | 0.0241 | 0.261 | 0.274 | 0.275 |
| 1-butene | g/g | 0.3230 | 0.9702 | 0.036 | 0.045 | 0.166 |
| c-2-butene | g/g | 0.2920 | 0.0000 | 0.309 | 0.291 | 0.236 |
| t-2-butene | g/g | 0.3550 | 0.0010 | 0.383 | 0.389 | 0.321 |
| isobutene | g/g | 0.0000 | 0.0000 | 0.000 | 0.000 | 0.000 |
| density | kg/m³ | 601 | 537 | 537 | 6 | 6 |
| Tem- | ° C. | 25.0 | 62.3 | 71.6 | 400.000 | 392 |
| perature | | | | | | |
| pressure | bar | 8.5 | 8.0 | 8.2 | 6.0 | 6.0 |

We claim:

1. A process for producing a 1-butene-containing C4-hydrocarbon stream (1-C₄⁼ stream) from a 1-butene- and 2-butene-containing C4-hydrocarbon stream (1- and 2-C₄⁼ feed stream) whose 1-butene content is lower than that of the 1-C₄⁼ stream, by
a) feeding the 1- and 2-C₄⁼ feed stream and a 1-butene- and 2-butene-containing C4-hydrocarbon stream (1- and 2-C₄⁼ recycle stream) whose 1-butene content is lower than that of the 1-C₄⁼ stream and which has been produced by means of step (b) below into a distillation column and taking off the 1-C₄⁼ stream and a 2-butene-containing C4-hydrocarbon stream (2-C₄⁼ stream) whose 1-butene content is lower than that of the 1- and 2-C₄⁼ feed stream and of the 1- and 2-C₄⁼ recycle stream from the distillation column (step a) and
b) producing the 1- and 2-C₄⁼ recycle stream from the 2-C₄⁼ stream by bringing the 2-C₄⁼ stream into contact with an isomerization catalyst which catalyzes the conversion of 2-butenes into 1-butene in a reaction zone (step b).

2. A process as claimed in claim 1, wherein a 1- and 2-C₄⁼ feed stream in which the ratio of 2-butenes to 1-butene is from 6:1 to 0.1:1 is used.

3. A process as claimed in either of the preceding claims, wherein a 1- and 2-C₄⁼ feed stream containing a maximum of 5% by weight of multiply unsaturated compounds or alkynes is used.

4. A process as claimed in any of the preceding claims, wherein a 1- and 2-C₄⁼ feed stream in which the content of butenes is from 30 to 100% by weight is used.

5. A process as claimed in any of the preceding claims, wherein the distillation column has from 30 to 80 theoretical plates and is operated at a reflux ratio of from 10 to 50.

6. A process as claimed in any of the preceding claims, wherein the 1-butene content, based on the sum of 1-butene and 2-butenes in the 1-C₄⁼ stream, is from 80 to 99.99%.

7. A process as claimed in any of the preceding claims, wherein the 1-C₄⁼ stream comprises
from 60 to 99.99% by weight of 1-butene and 2-butenes and
from 0.01 to 40% by weight of compounds selected from the group consisting of butadienes, isobutane, n-butane and isobutene.

8. A process as claimed in any of the preceding claims, wherein the 2-C₄⁼ stream is taken off in the lower fifth of the distillation column.

9. A process as claimed in any of the preceding claims, wherein the content of 2-butenes in the 1- and 2-C₄⁼ recycle stream has been reduced by from 5 to 30%, based on its content in the 2-C₄⁼ stream.

10. A process as claimed in any of the preceding claims, wherein a substream (C₄⁺) consisting essentially of 1-butene, 2-butenes, n-butane and hydrocarbons having 5 and more carbon atoms is taken off at the bottom of the distillation column.

11. A process as claimed in any of the preceding claims, wherein the temperature in the reaction zone of step b is from 200 to 500° C. and the pressure is from 1 to 20 bar.

12. A process as claimed in any of the preceding claims, wherein the conversion of 2-butenes into 1-butene, based on the content of 2-butenes in the 1- and 2-C₄⁼ feed stream, is from 70 to 99%.

13. A process as claimed in any of the preceding claims, wherein a 3-hexene-containing stream is prepared from the 1-C₄⁼ stream by bringing the 1- C₄⁼ stream into contact with a metathesis catalyst at from 20 to 350° C.

14. A process as claimed in any of the preceding claims, wherein the 1-C₄⁼ stream is freed of multiply unsaturated compounds and alkynes by subjecting it to a selective hydrogenation in the presence of a palladium-containing catalyst, in which virtually no conversion of 1-butene into 2-butenes occurs.
